(19) United States
(12) Patent Application Publication
(10) Pub. No.: US 2026/0241154 A1
(43) Pub. Date: Aug. 20, 2026

(54) APPLICATOR FOR PROVIDING MICRONEEDLES AND APPLICATOR PACKAGE COMPRISING SAME

(71) Applicant: CGBIO CO., LTD., Seoul (KR)

(72) Inventors: Chan Woo HONG, Incheon (KR); Hyun A PARK, Seongnam-si (KR)

(21) Appl. No.: 19/164,178

(22) PCT Filed: Sep. 19, 2023
(86) PCT No.: PCT/KR2023/014107

§ 371 Date: Sep. 10, 2025

(30) Foreign Application Priority Data
Mar. 13, 2023 (KR) 10-2023-0032286

(51) Int. Cl. A61M 37/00

(52) U.S. Cl. CPC A61M 37/0015; A61M 2037/0023; A61M 2209/06; A61M 2210/04

(57) ABSTRACT

The present disclosure relates to an applicator for providing a microneedle, and an applicator package including the same. According to one embodiment, microneedles can be inserted perpendicularly into the skin with constant force and speed due to a slide and a pressing part linked to and moving with the slide. The present disclosure aims to provide an applicator capable of vertically inserting a microneedle into the skin with a certain force and pressure, and an applicator package including the same.

### CROSS-REFERENCE TO RELATED APPLICATIONS

This application is the National Stage filing under 35 U.S.C. 371 of International Application No. PCT/KR2023/014107, filed on Sep. 19, 2023, which claims the benefit of Korean Patent Application No. 10-2023-0032286, filed on Mar. 13, 2023, the contents of which are all hereby incorporated by reference herein in their entirety.

### TECHNICAL FIELD The present disclosure relates to an applicator for providing a microneedle, and an applicator package including the same.

### BACKGROUND ART

Microneedles or micro-pins have small needle-shaped structures disposed therein, so they are used for the delivery of therapeutic agents and other substances through the skin and other surfaces. Microneedles are used in such a shape that they are pressed against a surface, particularly the skin, so that the therapeutic agents and other substances enter the underlying tissues sequentially or simultaneously through the surface such as the skin or the like. At this time, microneedles can penetrate the stratum corneum and deliver active ingredients into the body in a variety of ways. For example, hollow microneedles containing a reservoir to temporarily hold the active ingredients can penetrate the stratum corneum and deliver the active ingredients into the body, and since the microneedles are coated with the active ingredients, the microneedles can penetrate the stratum corneum and deliver the active ingredients into the body. In addition, soluble microneedles can penetrate the stratum corneum to deliver the active ingredients while being dissolved within the body.

Drug delivery using soluble microneedles is greatly affected not only by the direct mechanochemical properties of the microneedle including shape, physical properties, and solubility, but also by the application method such as the administration (insertion) state and administration time. Therefore, if the appropriate application method is not adopted for administration, not only may individual needles not be properly inserted into the dermal layer, but the fixed amount of drug may not be delivered. Furthermore, since there is a case that separate adhesive means such as pressure-sensitive adhesive sheets or adhesive paper are used in order to attach soluble microneedles to the skin and secure them in place for a long time, there have been difficulties in applying and attaching them to joints with a lot of movement, curved skin, or hairy skin.

To improve these problems, methods for administering microneedles using microneedle applicators have been disclosed, but there have been problems in that conventional microneedle applicators have complicated microneedle mounting methods and are vulnerable to damage or contamination of the microneedles when the microneedles are mounted on the applicators.

Furthermore, since a phenomenon that microneedles applied to applicators bend has been conventionally occurred, there has been a problem in that the microneedles are broken due to higher needle heights compared to cosmetic microneedles when used in pharmaceuticals or veterinary medicines.

In addition, there have been conventional problems in that the force and speed at which the microneedles were attached to the skin varied each time the applicator was used, and the microneedles were not attached vertically to cause them to break so that the drug could not be delivered completely.

Therefore, there is a need to develop an applicator capable of preventing damage and contamination of a microneedle and vertically inserting the microneedle into the skin at a consistent speed and pressure, and an applicator package including the same.

### DISCLOSURE

### Technical Problem

In order to solve the above problems, the present disclosure aims to provide an applicator capable of vertically inserting a microneedle into the skin with a certain force and pressure, and an applicator package including the same.

In addition, the present disclosure aims to provide an applicator in which the microneedle can be formed in a vertical direction without bending when a microneedle is separated from the microneedle package by the applicator, and an applicator package including the same.

In addition, the present disclosure aims to provide an applicator package that can prevent damage and contamination of the microneedle and improve usability.

In addition, the present disclosure aims to provide an applicator package that has a high level of reliability in the quality of the formed microneedle.

### Technical Solution

In order to solve the above problems, the present disclosure provides an applicator for providing a microneedle, which includes a slide and a pressing part that moves in conjunction with the slide, and inserts the microneedle into the skin in a vertical direction with a constant force and speed.

For example, the slide may move in a vertical direction with a constant force and speed along a linear guide formed along a length direction of the side portion of the applicator.

At this time, the slide may move a preset distance from the linear guide and be fixed at both ends of the preset distance.

Meanwhile, the pressing part may be at least partially inserted into the applicator and may move along a length direction of the applicator.

Furthermore, the microneedle may be disposed in front of the pressing part.

Furthermore, the applicator may have a structure that prevents rolling.

At this time, the applicator may include at least one of a polygonal structure having at least one corner, an oval structure, and a rolling-prevention member.

Furthermore, the present disclosure provides an applicator package including: the aforementioned applicator; and a microneedle package coupled to the applicator to provide the microneedle to the applicator.

At this time, the applicator may be operated by including: that when the applicator is inserted into the microneedle package, the slide moves to the rear of the applicator, the pressing part moves to the rear of the applicator in conjunction therewith, and the slide and the pressing part moving backward are fixed at a predetermined position; and that the slide and the pressing part fixed at the predetermined position move forward.

At this time, the microneedle package includes a needle protection part defining a space in which the microneedle is disposed and a case in which the needle protection part is disposed and into which the applicator is vertically inserted, the needle protection part includes a mold holder that is pushed by the applicator and moves forward and a skin contact part that is fastened to the front of the applicator, and the slide and the pressing part may be fixed at a predetermined position and the skin contact part may be fastened to the front of the applicator.

At this time, as a single operation of mounting the applicator on the microneedle package, an operation of fixing and loading the slide and the pressing part at a predetermined position, an operation of removing the mold holder, and an operation of fastening the skin contact part to the front of the applicator may be performed.

Furthermore, the case of the microneedle package may be provided with a guide means at the inner side, so that as the applicator is inserted into the case, the slide moves rearward by the guide means, and accordingly, the pressing part moves rearward, so that the pressing part does not have to be pushed in directly, thereby preventing contamination of the pressing part and the microneedle.

Meanwhile, the microneedle package may include a needle protection part defining a space in which the microneedle is disposed, and a case in which the needle protection part is disposed and into which the applicator is vertically inserted.

At this time, the needle protection part may include a mold holder that is pushed by the applicator and moves forward and a skin contact part that is fastened to the front of the applicator.

At this time, the mold holder may include a case fastening part that is secured to a predetermined position within the case.

At this time, the case fastening part may be pressed by the applicator, thereby moving the mold holder forward.

At this time, the case fastening part may be provided with three pressed regions that are pressed by the applicator, and the mold holder is pressed by the applicator such that the mold holder has a surface perpendicular to the applicator, thereby forming the microneedle vertically.

Additionally, the slide may be moved backward by a guide member provided in the microneedle package.

At this time, the applicator further includes a linear guide provided to guide the slide to move in a vertical direction with a constant force and speed, and the guide member may be inserted into the linear guide as the applicator is inserted into the microneedle package.

### Advantageous Effects

An applicator and applicator package according to one embodiment of the present disclosure can vertically insert a microneedle into the skin with a constant force and pressure.

In addition, an applicator and applicator package according to one embodiment of the present disclosure can vertically insert a microneedle into the skin with little force.

In addition, an applicator and applicator package according to one embodiment of the present disclosure can form a microneedle in a vertical direction on the front surface of the applicator without the microneedle being bent.

In addition, an applicator and applicator package according to one embodiment of the present disclosure can prevent contamination and damage to the microneedle attached to the front surface of the applicator.

### DESCRIPTION OF DRAWINGS

FIGS. 1 and 2 are for explaining an applicator package according to an embodiment of the present disclosure, wherein FIG. 1 shows the applicator package, and FIG. 2 shows the microneedle package disclosed in FIG. 1.

FIGS. 3 and 4 show a state in which an applicator according to an embodiment of the present disclosure is in contact with the skin, wherein FIG. 4 shows a front surface of the applicator according to FIG. 3.

FIGS. 5 and 6 show a form of a microneedle introduced into the skin, wherein FIG. 5 shows a conventionally manufactured microneedle, and FIG. 6 shows a microneedle manufactured according to one embodiment of the present disclosure.

FIGS. 7, 8, and 9 show embodiments of forming and separating a microneedle from a mold according to an embodiment of the present disclosure.

FIGS. 10 and 11 show a mold holder and a skin contact part according to an embodiment of the present disclosure.

FIGS. 12 and 13 show embodiments of a microneedle cartridge and a skin contact part separated therefrom according to an embodiment of the present disclosure.

FIGS. 14, 15, 16, 17, 18, and 19 show embodiments in which a skin contact part according to an embodiment of the present disclosure is mounted on an applicator and separated from a microneedle package.

FIGS. 20, 21, 22, and 23 show embodiments in which an applicator according to an embodiment of the present disclosure contacts the skin, a microneedle is fired, and then is separated from the skin.

### MODE FOR DISCLOSURE

Hereinafter, embodiments of the present disclosure will be described in detail. However, the present disclosure is not limited to the embodiments disclosed below and may be implemented in various other forms, and these embodiments are provided solely to ensure the complete disclosure of the present disclosure and to more fully convey the content of the present disclosure to those skilled in the art.

When one element is referred to as being positioned “above” or “below” another element in this specification, this includes both a meaning that the one element is directly positioned “above” or “below” the other element, or a meaning that additional elements can be interposed between those elements.

In this specification, the terms “upper” and “lower” are relative concepts established from the observer's perspective, and if the observer's perspective changes, “upper” may mean “lower,” and “lower” may mean “upper.”

In this specification, the terms “top” and “bottom” are relative concepts established from the observer's perspective, and if the observer's perspective changes, “top” may mean “bottom,” and “bottom” may mean “top.”

In this specification, “front direction” or “front” refers to the direction in which the applicator is attached to the skin, “rear direction” or “rear” refers to the opposite direction, and “front” refers to the surface formed in the front, and “rear” refers to the surface formed in the opposite direction.

Unless otherwise specifically limited in this specification, “length direction” refers to the direction in which the applicator approaches the skin or enters the case, and “thickness direction” refers to the direction perpendicular to the “length direction”.

Unless otherwise specifically limited in this specification, “vertical direction” may refer to a direction in which 90 degrees are formed based on the front surface of the skin contact part.

The applicator referred to in this specification includes a state in which at least some components of a microneedle package are coupled to the applicator.

The applicator package referred to in this specification includes a state in which at least some components of the applicator and the microneedle package are included, and includes a state in which the applicator and the microneedle package are separated.

The microneedle package referred to in this specification includes a microneedle cartridge and a case.

The microneedle cartridge referred to in this specification includes a needle protection part and a microneedle, and may additionally include a mold depending on the embodiment of forming the microneedle.

The needle protection part referred to in this specification includes a mold holder and a skin contact part, and the mold holder and the skin contact part may be integrally formed or structurally separated from each other.

The following is a detailed explanation with reference to the drawings.

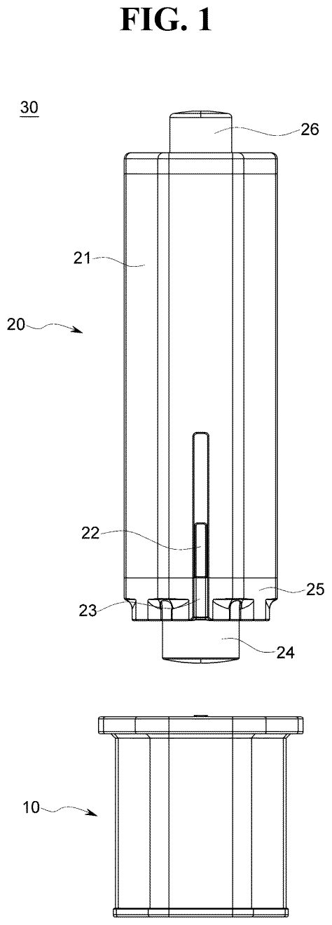

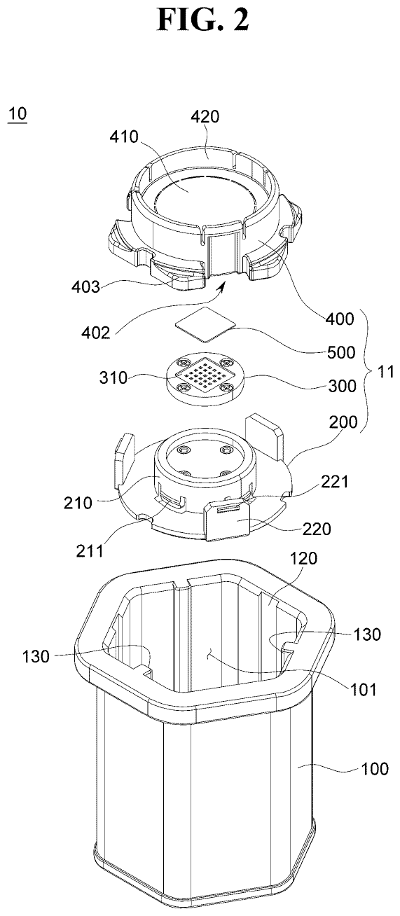

FIGS. 1 and 2 are for explaining an applicator package according to an embodiment of the present disclosure, wherein FIG. 1 shows the applicator package, and FIG. 2 shows the microneedle package disclosed in FIG. 1.

Referring to FIGS. 1 and 2, the applicator package includes a microneedle package 10 and an applicator 20.

The detailed structure of the microneedle package 10 will be described later with reference to FIG. 2.

The applicator 20 may include an applicator body 21, a slide 22, a pressing part 24, a skin contact part fastening part 25, and a shooting button 26.

The applicator body 21 allows the user to grip it and insert its distal end into the microneedle package 10, and may have a form in which it is extended by a predetermined length in the length direction.

The applicator body 21 may be applied with a structure to prevent rolling when positioned on a treatment table or the like prior to the procedure.

For example, the applicator body 21 may include at least one of a polygonal structure having at least one corner, an oval structure, and a rolling-prevention member. For example, the rolling-prevention member may include a protrusion (not shown) provided on the side portion of the applicator body 21.

A slide 22 may be provided at the distal end of the applicator body 21.

The slide 22 moves in conjunction with the pressing part 24, thereby performing functions of vertically attaching the microneedle 500 to the front surface of the applicator 20, causing the pressing part 24 to move in a vertical direction toward the skin, and moving the pressing part 24 toward the skin with a constant force.

For example, the slide 22 may move a predetermined distance from the end of the body 21.

For example, the slide 22 can move up and down along a linear guide 23 formed in the length direction on the side surface of the applicator body 21, and may be fixed at a predetermined position.

For example, the linear guide 23 may be provided in the applicator body 21. For example, the linear guide 23 may be provided on the side portion, front, or inside of the body 21. For example, the linear guide 23 may be a groove form provided in the length direction on the side portion thereof. For example, when performing an operation of inserting the applicator 20 into the case 100 of the microneedle package 10, the protrusion piece 130 provided in the case 100 is inserted into the linear guide 23, so that the rotation of the applicator 20 is restricted and the applicator moves in the length direction, thereby allowing the applicator 20 to be inserted vertically into the microneedle 500. For example, a plurality of linear guides 23 of the present disclosure may be provided, thereby preventing the applicator body 21 from rotating when performing an operation of inserting the applicator body 21 into the case 100 of the microneedle package 10.

For example, an operation of pushing the slide 22 backward by a protrusion piece 130 provided inside the case 100 may be performed.

For example, as the applicator 20 is inserted into the case 100 of the microneedle package 10, the slide 22 may be pushed backward, and the slide 22 may be fixed in a state of being pulled backward at a depth where the skin contact part 400 and the microneedle 500 are mounted at the end of the applicator body 21 within the case 100.

For example, one or more slides 22 may be provided, and for example, three slides may be provided in different regions on the side surface of the applicator 20, with protrusion pieces 130 provided at positions corresponding to the slides 22, allowing the applicator 20 to be stably inserted into the case 100 along a vertical direction with respect to the microneedle 500.

For example, the distance at which the slide 22 moves in the linear guide 23 is controlled to be constant, and as the slide 22 moves forward at a preset constant distance with a constant force and speed, the pressing part 24, which moves in conjunction with the movement of the slide 22, moves forward at a preset constant distance with a constant force and speed, allowing the microneedle 500 to be inserted into the skin at a constant force and speed.

At least a portion of the pressing part 24 may be provided within the applicator body 21 and may move in the length direction along a space formed within the applicator body 21.

For example, the pressing part 24 may be applied with a structure of moving so as to protrude forward of the applicator body 21, or may be applied with a structure of moving the microneedle 500 within the applicator body 21 so as to protrude forward of the applicator body 21.

For example, the pressing part 24 may be driven by a firing means disposed within the applicator body 21.

For example, the slide 22 and the pressing part 24 may be driven by the firing means through physical or electrical linkage to move in the length direction.

For example, the firing means may include those that are mechanically or electrically driven using a spring, hydraulic pressure, a driving motor, a gear motion, or the like, but the present disclosure is not limited thereto.

For example, the firing means may be driven to move forward at a preset constant force and speed, allowing the pressing part 24 to move forward at a constant force and speed. Accordingly, the microneedle 500 may be inserted into the skin at a constant force and speed.

The skin contact part fastening part 25 is formed at the distal end of the applicator body 21 and refers to the region fastened to the skin contact part 400.

For example, the skin contact part fastening part 25 may have a structure that allows the skin contact part 400 to be inserted and then fitting-coupled. For example, the skin contact part fastening part 25 and the skin contact part 400 may be stably fastened by coupling through a female-male structure, elastic bonding, or the like, but a structure that is detachable again after use may be applied.

The shooting button 26 may be a start button that causes the pressing part 24 to advance forward.

For example, the shooting button 26 may be provided by being protruded to the outside of the applicator body 21, and for example, the shooting button 26 may be provided on at least one of the side and rear of the body 21.

For example, the shooting button 26 may be a communication device, not a physical structure, and may also be remotely controlled.

The driving of the shooting button 26 is linked to the operation of the firing means, so that the firing means is operated by the driving of the shooting button 26, and accordingly, the pressing part 24 and the slide 22 linked to the firing means may move forward.

For example, the shooting button 26 may be divided into a loaded state and an operating state. In one example, when the applicator 20 is inserted into the case 100 of the microneedle package 10 and reaches a preset position, the slide 22 moves backward by a guide member, for example, a protrusion piece 130, and reaches a preset position, and at the preset predetermined position, the movement of the slide 22 stops, and at the same time, the shooting button 26 enters a loaded state.

For example, if the user touches or applies pressure to the shooting button 26 when the shooting button 26 is in the loaded state, the pressing part 24 can move forward with a preset force and speed. At this time, the movement length of the slide 22 may also be controlled so that the end of the slide 22 does not impact the skin when the slide 22 moves forward together with the pressing part 24.

Referring to FIG. 2, the microneedle package 10 includes a case 100 and a microneedle cartridge 11.

The case 100 may have an open one surface and a corresponding closed other surface, may be in the form of a cup with the one surface and the other surface connected in the length direction, and may have a case opening 101 formed in the one surface. The microneedle cartridge 11 may be inserted into and mounted within the case 100 through the case opening 101.

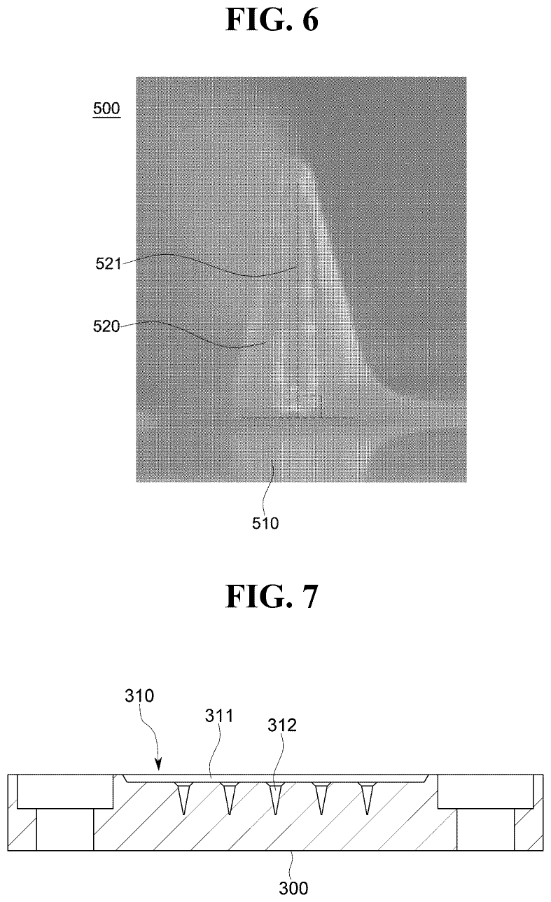

For example, the case 100 may be formed to a predetermined length so that the applicator 20 may be inserted and separated by moving to the predetermined length, and at this time, the movement of the applicator 20 may be guided so that it moves in one direction, thereby ensuring that the microneedle 500 is formed in a vertical direction without bending when the microneedle 500 is separated from the microneedle package 10 by the applicator 20. At this time, the meaning of being formed in a vertical direction further includes at least one of: that the needle 520 is formed in a form of extending the center line 521 of the needle 520 vertically from the base 510 as illustrated in FIG. 6; and that the needle 520 is formed to gradually narrow the cross-sectional area at a predetermined ratio as it extends from the base 510, and the same applies to the following description.

For example, a guide means guiding the insertion of the applicator 20 may be provided in the inner side of the case 100. As an example of the guide means, a protrusion piece 130 in a form of extending from the inner side of the case 100 with a predetermined length may be provided. As an example of the guide means, the protrusion piece 130 performs functions of guiding a vertical directional insertion of the applicator 20 and, at the same time, applying pressure to the slide 22 of the applicator 20 to stably push up the slide 22. In addition, the protrusion piece performs a function of preventing the applicator 20 from rotating when inserting it into the case 100.

The microneedle cartridge 11 may include a needle protection part and a microneedle 500, and may further include a mold 300 when the microneedle 500 is molded and formed.

The needle protection part is not particularly limited in form or function as long as a microneedle 500 is disposed.

For example, the needle protection part may be comprised of a single member or multiple members.

For example, the needle protection part may be a needle protection part 12 including a mold holder 200 and a skin contact part 400 as illustrated in FIG. 10.

A microneedle 500 may be formed in the mold holder 200, and for example, a mold 300 may be disposed in the mold holder 200.

A mold mounting part 210 for mounting the mold 300 may be provided at the center of the mold holder 200.

The mold mounting part 210 may include a form corresponding to the shape of the mold 300 so that the mold 300 may be mounted and secured thereon. For example, the mold mounting part 210 may include a recessed region for seating the mold 300 and fixing its movement.

When the mold 300 is coupled to and then separated from the applicator 20 in the applicator package 30, the mold 300 remains in the mold holder 200, and the microneedle 500 may be stably vertically detached from the mold 300. For example, the microneedle 500 may be detached from the mold 300 together with the adhesive means 410.

For example, the mold 300 may be a single member manufactured integrally with the mold holder 200, and the term may be simply distinguished from the mold holder 200 to refer to the region where the microneedle 500 is formed.

For example, the mold 300 may be a member that can be separated and fastened to the mold holder 200. The fastening method may be applied in various ways, such as a fastening means such as a screw, a member with magnetism, an adhesive means, and a fitting coupling by elasticity.

The mold mounting part 210 may have a predetermined area and have a form of extending backward from the mold holder 200. At this time, an elastic piece 211 protruding in the thickness direction may be formed on the side portion of the mold mounting part 210. The elastic piece 211 is applied to fasten and separate the mold holder 200 from the skin contact part 400.

For example, when the microneedle cartridge 11 is configured in a state in which the mold holder 200 and the skin contact part 400 coupled, at least a portion of the mold mounting part 210 may be provided in a state in which it is inserted into the skin contact part 400. At this time, the elastic piece 211 may be fitting-coupled into the skin contact part 400 to prevent the skin contact part 400 from being separated from the mold holder 200 within the case 100 constituting the microneedle package 10. Meanwhile, the fitting coupling of the elastic piece 211 to the inner side of the skin contact part 400 may be released by the withdrawal force that separates the applicator 20 from the microneedle package 10 after the applicator 20 is coupled, thereby separating the skin contact part 400 from the mold holder 200.

Accordingly, when attaching the microneedle 500 to the front of the applicator 20 or the front surface of the pressing part 24 of the applicator 20, not only does the user not have to use their hands or equipment, but also the microneedle 500 can be introduced to the front of the applicator 20 or the front surface of the pressing part 24 of the applicator 20 in a state in which the microneedle 500 is blocked from the external environment, thereby preventing contamination and damage to the microneedle 500.

In addition, even before use, the microneedle 500 may be protected within the case 10 and blocked from the external environment, thereby preventing contamination and damage to the microneedle 500, and at the same time, enhancing convenience of use. Furthermore, organic coupling with other components of the present disclosure allows the applicator 20 to be inserted in a vertical direction, and the microneedle 500 can be formed in a vertical direction without bending.

The mold holder 200 may have a case fastening part 220 formed at the end in the thickness direction.

For example, the case fastening part 220 may have a form in which multiple case fastening parts 220 extend backward. Accordingly, in the present disclosure, the mold holder 200 in a state in which its position is fixed within the case 100, can move forward by an operation of allowing the applicator 20 to simultaneously press multiple case fastening parts 220 forward. At this time, if only one or some of these are pressed by the applicator 20, the position of the mold holder 200 may remain a state of being fixed within the case 100, or the microneedle 500 may not be separated from the mold holder 200.

Therefore, the mold holder 200 of the present disclosure may be vertically moved forward by the applicator 20.

The mold holder 200 may be pressed by the applicator 20 in a state in which the mold holder 200 is stably maintained so that it has a surface perpendicular to the applicator 20.

For example, the mold holder 200 moves forward by pressing the case fastening part 220 by the applicator 20, and the case fastening part 220 may be provided with three pressed regions that are pressed by the applicator 20. The pressed regions include both points, and surfaces having a predetermined flat surface.

For example, a structure which is formed by forming the same angle based on the center of the mold holder 200 and pressed at the same time may be applied to the three pressed regions.

For example, a structure which is simultaneously pressed by the applicator 20 may be applied to the three pressed regions.

For example, three case fastening parts 220 may be provided, and each case fastening part 220 may be provided with one pressed region, allowing the skin contact part fastening part 25 to be stably seated on three surfaces of the fastening parts 220.

That is, the mold holder 200 of the present disclosure includes the three pressed regions simultaneously pressed by the applicator 20 so that the mold holder 200 is pressed by the applicator 20 while stably maintaining a state in which the mold holder 200 has a vertical surface, thereby vertically forming the microneedle 500, and preventing damage to the microneedle 500.

In addition, the microneedle 500 may be vertically separated from the mold holder 200 by an operation of vertically moving the mold holder 200 forward. The microneedle 500 may be vertically separated from the mold holder 200 and attached to the front of the applicator 20 in a vertical direction.

For example, the case fastening part 220 may have a form that extends rearward by a predetermined length. At this time, a guide groove 120 into which the case fastening part 220 may be guided and inserted is formed in the inner side of the case 100, so that the case fastening part 220 may be inserted into the inner side of the case 100 while maintaining a consistent direction.

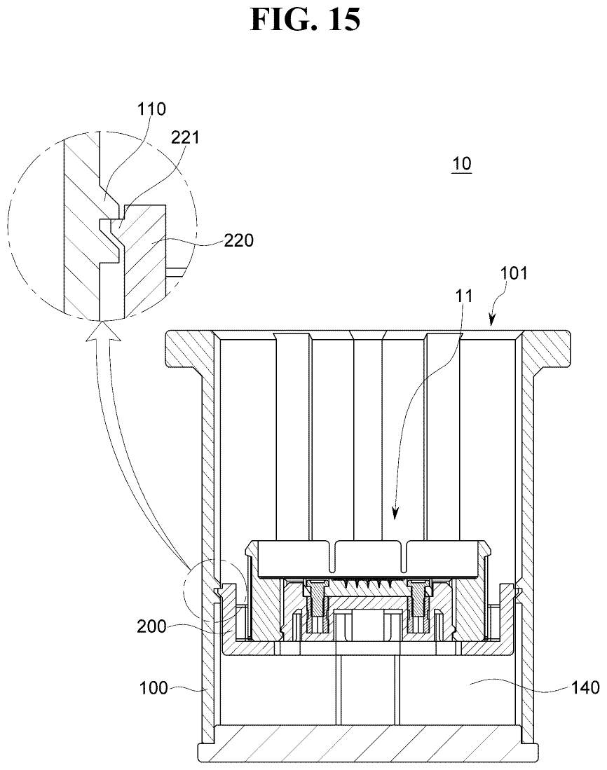

For example, the case fastening part 220 may be provided with an outwardly protruding protrusion 221, and a catching projection (110 in FIG. 15) into which the protrusion 221 may be inserted may be provided on the inner side of the case 100, such that the case fastening part 220 may be fixed in a state in which rotation and longitudinal movement are limited at a predetermined position within the case 100. This will be described in detail with reference to FIG. 15 described later.

The mold 300 is for forming a microneedle 500, and a microneedle forming part 310 is provided at the center of the mold 300.

The microneedle forming part 310 may be provided with a plurality of grooves having a predetermined form so that the microneedle 500 is formed or mounted thereon.

For example, a drug may be filled into the microneedle forming part 310 and then cured to form the microneedle 500. Accordingly, the microneedle 500 may be stably formed in the mold, preventing the microneedle 500 from being exposed to the outside, and safely protecting the microneedle 500 from impact. This will be described in detail with reference to FIGS. 7, 8, and 9 described later.

For example, the mold 300 may be manufactured from a metal material or polymer material, and for example, a plastic material may be applied. Accordingly, the mold 300 may be manufactured at an inexpensive unit cost, sterilization-treated easily, and the microneedle 500 formed in the microneedle forming part 310 may be easily separated from the microneedle forming part 310.

The skin contact part 400 is for guiding the forward insertion of the applicator 20, and disposed on the rear surface of the mold holder 200.

Since the skin contact part 400 has a front surface contacting the skin when the applicator package 10 is operated formed so that the skin contact part 400 pulls the skin while pressing against the skin, the microneedle 500 can be vertically inserted into the skin even with a small amount of force, thereby consequently vertically inserting the microneedle 500 with a constant force and pressure.

At this time, a friction force providing part (not shown) may be included on the front surface contacting the skin in the skin contact part 400, and for example, the friction force providing part may enhance the skin pulling effect by including irregularities.

The skin contact part 400 may be provided with an adhesive means 410 and a guide member 420 formed to surround the adhesive means 410 and extended a predetermined length rearward.

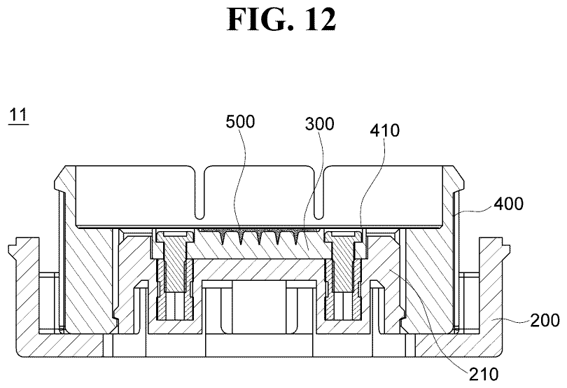

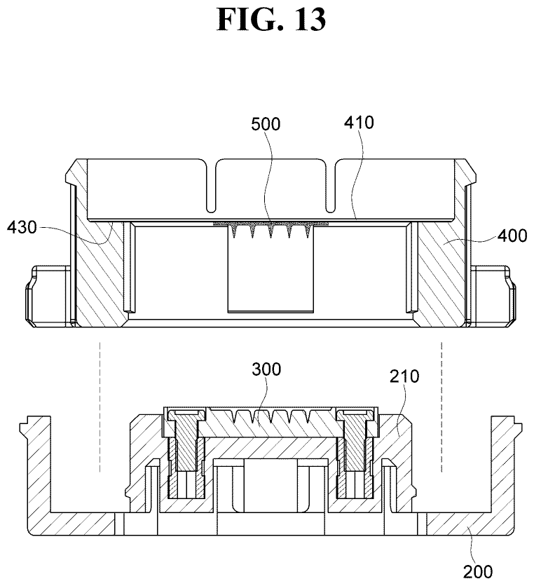

The skin contact part 400 may be fastened to and separated from the mold holder 200, and may be fastened to the applicator 20 and separated from the mold holder 200 together with the microneedle 500. This will be described in detail with reference to FIGS. 12 and 13 described later.

The skin contact part 400 is provided in a state in which it is coupled to the mold holder 200, but when applied to the applicator, the microneedle 500 is separated from the mold holder 200 in a state in which the microneedle 500 is attached to the skin contact part 400, thereby preventing contamination and damage to the microneedle 500 before use and improving use convenience. Alternatively, the skin contact part 400 may be disposed to have an overlapping region within the case 100 without being physically coupled to the mold holder 200.

For example, when the skin contact part 400 and the mold holder 200 are fastened, the front surface of the adhesive means 410 located at the central portion of the skin contact part 400 and the rear surface of the microneedle 500 formed in the mold 300 mounted on the mold mounting part 210 come into contact with each other. Accordingly, the microneedle 500 formed in the mold 300 in the microneedle package 10 can be isolated from the external environment by the mold 300, the mold holder 200, and the adhesive means 410, thereby preventing contamination and damage. This will be described in detail with reference to FIGS. 12 and 13 described later.

In the microneedle package 10 of the present disclosure, the mold mounting part 210, the mold 300, the microneedle 500, and the adhesive means 410 may be each continuously disposed in the length direction in a state in which they have a front surface and a rear surface with a predetermined area in the thickness direction, and the applicator 20 is inserted into the case 100 in the length direction, thereby having a structure in which the rear surface of the microneedle 500 is firmly attached to the front surface of the adhesive means 410, and the rear surface of the adhesive means 410 is firmly attached to the front surface of the applicator pressing part.

In this way, the present disclosure provides a state in which the skin contact part 400 is disposed to have an overlapping region within the case 100 in a state in which the skin contact part 400 is coupled to the mold holder 200 or without physical coupling, and when applied to the applicator 20, the skin contact part 400 is separated from the mold holder 200 in a state in which the microneedle 500 is adhered to the adhesive means 410, thereby preventing contamination and damage to the microneedle 500 before use and improving use convenience.

In addition, the mold holder 200 and the skin contact part 400 of the present disclosure are installed within a case 100 that extends in the length direction to have a predetermined length, and the mold holder 200 is fixed within the case 100, but a structure that is not fixed within the case 100 may be applied to the skin contact part 400, thereby easily separating the skin contact part 400 from the case 100 by the applicator 20.

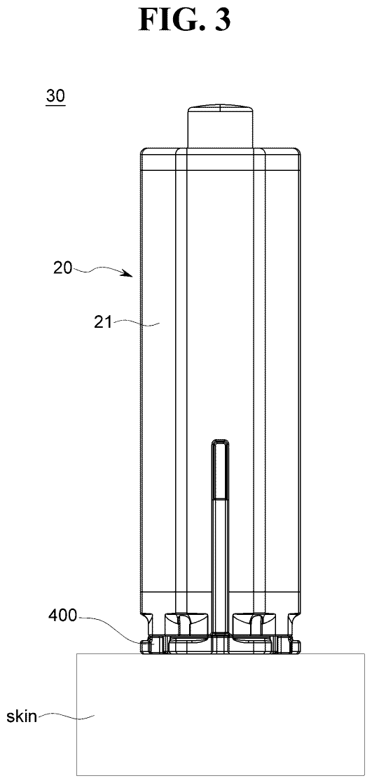

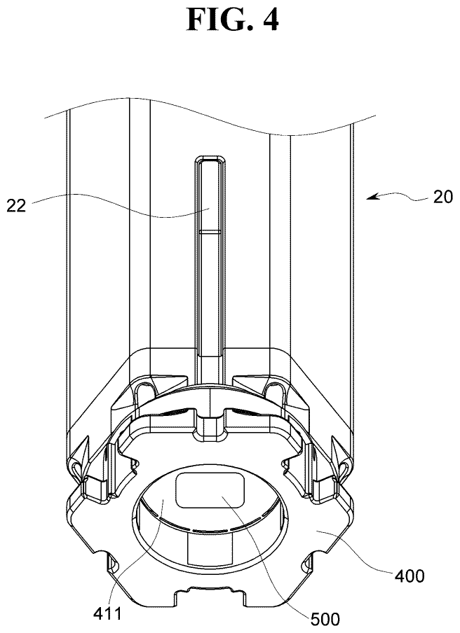

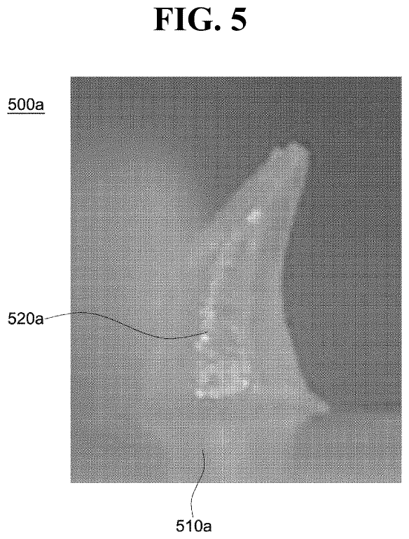

FIGS. 3 and 4 show a state in which an applicator according to an embodiment of the present disclosure is in contact with the skin, wherein FIG. 4 shows a front surface of the applicator according to FIG. 3, and FIGS. 5 and 6 show a form of a microneedle introduced into the skin, wherein FIG. 5 shows a conventionally manufactured microneedle, and FIG. 6 shows a microneedle manufactured according to one embodiment of the present disclosure.

Referring to FIGS. 3 and 4, the applicator 20 may be provided in a state in which a skin contact part is attached to the front of the applicator 20.

At this time, referring to FIGS. 1 and 2 together, the applicator 20 may be provided in a state in which a microneedle 500 is attached to the front of the pressing part 24. For example, the microneedle 500 may be a form in which it is attached directly to the front surface of the pressing part 24, or attached to the front of a needle attachment part 411 disposed on the front surface of the pressing part 24.

The skin contact part 400 may be coupled to the applicator in a form that surrounds the side portion of the pressing part 24, and may be formed to have a predetermined area on the front surface. For example, when the longest length from the center of the front surface of the pressing part 24 to the lateral end of the front surface of the pressing part 24 is a, and the longest length from the center of the front surface of the pressing part 24 to the lateral end of the front surface of the skin contact part 400 is b, b may be in the range of 1.5 to 3 times a. For example, the area of the front surface of the skin contact part 400 may be 0.5 to 5 times the area of the front surface of the pressing part 24.

Accordingly, when the applicator 20 of the present disclosure comes into contact with the skin, the effect of laterally pulling the skin is generated while the skin is being pressed by the front surface of the skin contact part 400 as well as the front surface of the pressing part 24. In the related art, when the microneedle 500 is directly introduced or there is no skin pulling effect by the skin contact part 400, the microneedle 500 should be introduced into relatively thick skin, so the microneedle 500 should be inserted into the skin with a relatively strong force, and accordingly as illustrated in FIG. 5, the microneedle 500 breaks or bends, making vertical insertion difficult. In contrast, since the applicator 20 of the present disclosure introduces the microneedle 500 in a state in which the skin is pulled by the front surface of the skin contact part 400 as well as the front surface of the pressing part 24, the microneedle 500 can be introduced into a relatively thin skin to allow the microneedle 500 to be vertically inserted into the skin with relatively little force, thereby allowing the microneedle 500 to be inserted vertically into the skin as illustrated in FIG. 6.

In addition, the skin contact part 400 may further include a friction force providing member on the front surface. The friction force providing member may include, for example, irregularities, etc. Accordingly, the skin pulling effect can be further enhanced by the front surface of the skin contact part 400.

Hereinafter, a method for forming the microneedle 500 and a method for applying the skin contact part 400 to the applicator 20 will be described.

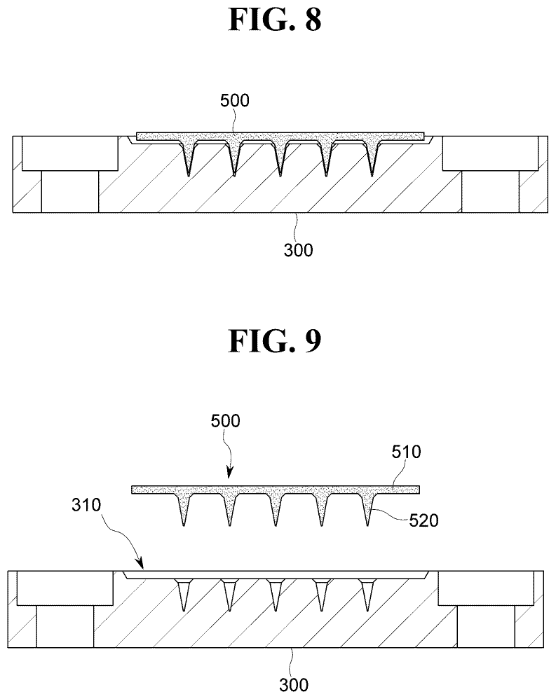

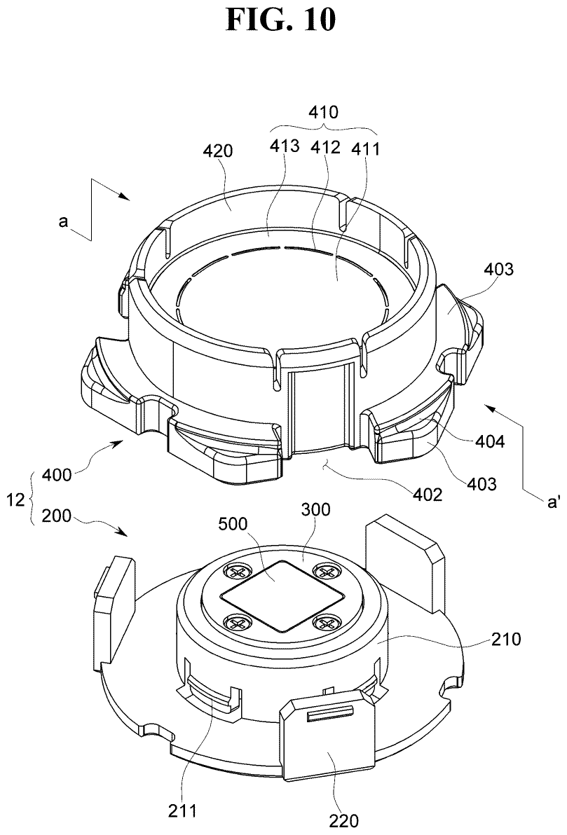

FIGS. 7, 8, and 9 show embodiments of forming and separating a microneedle from a mold according to an embodiment of the present disclosure, FIG. 7 shows a state before a microneedle 500 is formed in a microneedle forming part 310, FIG. 8 shows a state in which a microneedle 500 is formed in a microneedle forming part 310, and FIG. 9 shows an embodiment in which a microneedle 500 is separated from a microneedle forming part 310.

Referring to FIG. 7, a microneedle forming part 310 may be provided in a mold 300.

For example, the microneedle forming part 310 may include a base forming part 311 formed by recessing a predetermined depth in the central portion of the mold 300, and a plurality of needle forming parts 312 extending forward from the base forming part 311 by a predetermined depth.

Referring to FIGS. 8 and 9, a microneedle 500 may be formed in the microneedle forming part 310.

For example, when a drug for manufacturing a soluble microneedle 500 is applied to the microneedle forming part 310, the drug may be filled into the microneedle forming part 310, thereby forming a base 510 in the base forming part 311 and forming needles 520 in the needle forming part 312.

Referring to FIG. 9, since the entire area of the microneedle 500 together with an adhesive means (not shown) is simultaneously separated in the length direction from the mold 300, the needles 520 may be formed vertically on the base 510 without bending.

In this way, the microneedle 500 of the present disclosure may be prevented from being exposed to the external environment and safely protected from impact before the microneedle 500 is separated from the mold 300 using the applicator 20, and at the same time, the needles 520 may be formed vertically on the base 510.

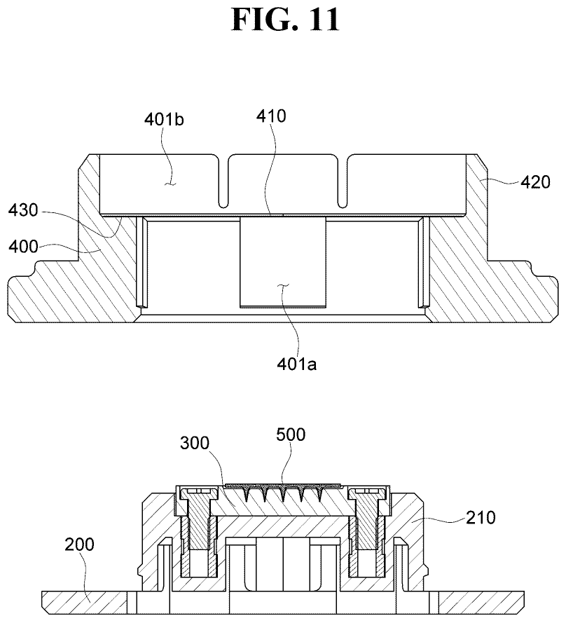

FIGS. 10 and 11 show a mold holder and a skin contact part according to an embodiment of the present disclosure, and FIG. 11 shows a cross-section taken along the aa′ direction in FIG. 10. In addition, FIGS. 12 and 13 show embodiments of a microneedle cartridge and a skin contact part separated therefrom according to an embodiment of the present disclosure, FIG. 12 shows one embodiment of a microneedle cartridge, and FIG. 13 shows that the skin contact part and the microneedle are separated from the microneedle cartridge of FIG. 12.

Referring to FIGS. 10, 11, 12, and 13, the skin contact part 400 is provided in a state in which it is seated on the mold holder 200, and the skin contact part 400 may be separated from the mold holder 200 by the insertion motion of the applicator 20.

The skin contact part 400 may be provided with a guide member 420 defining a space that guides the approach of the applicator 20. For example, the guide member 420 may be formed by being extended a predetermined length backward from the skin contact part 400. For example, the distal end of the applicator 20 may be guided to surround the guide member 420. For example, the guide member 420 may be composed of a plurality of segments separated by predetermined length units. Accordingly, the applicator 20 is guided by the guide member 420 to contact in a vertical direction the rear surface of the microneedle 500 disposed in the mold 300 and the rear surface of the adhesive means 410, and when the distal end of the applicator 20 is inserted into the skin contact part 400, a predetermined elastic force by the guide member 420 is applied so that the applicator 20 enters in the forward direction until the moment when the applicator and the adhesive means 410 come into contact, and the adhesive means 410 may be fastened to the distal end of the applicator 20 with the predetermined elastic force.

The skin contact part 400 may be provided with a plurality of tongue parts 403 protruding outward in the thickness direction from the guide member 420.

A case fastening part guide part 402 into which the case fastening part 220 can be inserted may be provided between the plurality of tongue parts 403. Accordingly, the skin contact part 400 may be inserted and withdrawn in a vertical direction from the rear surface of the mold holder 200 so that the needles 520 may be formed vertically from the base 510. In addition, the case fastening part 220 may be inserted into the case fastening part guide part 402 so that the rotation of the skin contact part 400 may be restricted.

The front surface of the tongue part 403 is a surface that comes into contact with the skin when the microneedle 500 is fired onto the skin, and may be formed to have a predetermined area so that the position and direction of the applicator 20 on the skin can be stably set.

A tongue part groove 404 may be provided on the tongue part 403. The distal end of the applicator 20 may be fitting-coupled into the tongue part groove 404, and as the applicator 20 is withdrawn, the skin contact part 400 may be separated from the mold holder 200.

The skin contact part 400 may be provided with a step 430 formed by extending a predetermined length inward from the guide member 420.

The step 430 may protrude inward of the guide member 420 at a predetermined height of the skin contact part 400 and extend forward of the skin contact part 400 to define a region into which the mold holder 200 is inserted.

An adhesive means 410 may be seated on the step 430, and in one embodiment, a thickness directional distal end of the adhesive means 410 may be adhered to the step surface of the step 430.

The adhesive means 410 may include a needle attachment part 411, a skin contact part attachment part 413, and an adhesive means cutting line 412.

The needle attachment part 411 is part that comes into contact with the microneedle 500, and may have adhesive force on the front surface. Accordingly, the rear surface of the microneedle 500 may be adhered to the front surface of the needle attachment part 411.

When the microneedle 500 is fired into the target area, for example, the skin, the needle attachment part 411 may be separated from the skin contact part attachment part 413.

In addition, when the microneedle 500 attached to the adhesive means 410 is brought into contact with the skin using an applicator, the adhesive means 410 may be adhered to the skin by the adhesive force of the region of the needle attachment part 411 where the microneedle 500 is not attached.

The skin contact part attachment part 413 may include a region attached to the step 430 in the adhesive means 410. The adhesive means 410 may be fixed to the skin contact part 400 by the skin contact part attachment part 413.

The adhesive means cutting line 412 may be defined as an empty region formed between the needle attachment part 411 and the skin contact part attachment part 413. Accordingly, when the applicator 20 presses the needle attachment part 411, the needle attachment part 411 may be easily separated from the skin contact part attachment part 413 along the adhesive means cutting line 412.

The skin contact part 400 may be formed with skin contact part penetration parts 401a and 401b penetrating the lower and upper portions, and the skin contact part penetration parts 401a and 401b may have different vertical direction cross-sectional areas based on the adhesive means 410. For example, in the skin contact part penetration part 401b located at the rear of the skin contact part 400, a vertical direction cross-sectional area is defined by the aforementioned guide member 420, and the skin contact part penetration part 401a located at the lower portion of the skin contact part 400 is defined as an area narrower than the skin contact part penetration part 401b by the step 430.

At this time, the pressing part 24 of the applicator 20 is inserted along the adhesive means housing penetration part 401b and presses the needle attachment part 411 to attach the rear surface of the microneedle 500 to the front surface of the needle attachment part 411, and when the pressing part returns to the rear in a state in which the rear surface of the needle attachment part 411 is attached to the pressing part, the needle attachment part 411 is separated from the skin contact part attachment part 413 while the cutting line 412 is being cut.

Referring to FIGS. 12 and 13, a structure in which the mold holder 200 and the skin contact part 400 are coupled to and separated from each other in the microneedle cartridge 11 may be applied. That is, the microneedle cartridge 11 may be provided in a state in which the mold holder 200 and the skin contact part 400 are coupled or in a state in which they have an overlapping region without physical coupling, and a structure may be applied in which the skin contact part 400 and the microneedle 500 are separated from the microneedle cartridge 11 as the skin contact part 400 is separated from the mold holder 200 by the applicator 20.

The microneedle cartridge 11 may be provided in a state in which the mold holder 200 and the skin contact part 400 are coupled to each other. Accordingly, the adhesive means 410 covers the rear of the microneedle 500 formed in the mold 300 fixed to the mold mounting part 210, thereby blocking the microneedle 500 from being exposed to the outside. Accordingly, contamination and damage to the microneedle 500 are prevented.

Referring to FIG. 12, the microneedle cartridge 11 may include a state in which a mold holder 200 and a skin contact part 400 are coupled. Referring to FIG. 13, the mold holder 200 and the skin contact part 400 may be separated from each other by the withdrawal action of the applicator 20. At this time, the mold 300 fixed to the mold mounting part 210 remains in a state in which it is fixed to the mold mounting part 210, and the microneedle 500 formed in the mold 300 may be separated from the mold 300 in a state in which the microneedle 500 is attached to the adhesive means 410 of the skin contact part 400.

For example, when the applicator 20 is fastened to the microneedle cartridge 11, the skin contact part 400 is coupled to the applicator 20, and when the applicator 20 is separated from the microneedle cartridge 11, the skin contact part 400 may be separated from the mold holder 200. Accordingly, the microneedle 500 attached to the adhesive means 410 may also be vertically extracted from the mold 300 and safely separated without damaging the microstructure of the microneedle 500. In addition, since the operator does not need to contact the region where the microneedle 500 is mounted, damage and contamination of the microneedle 500 are prevented.

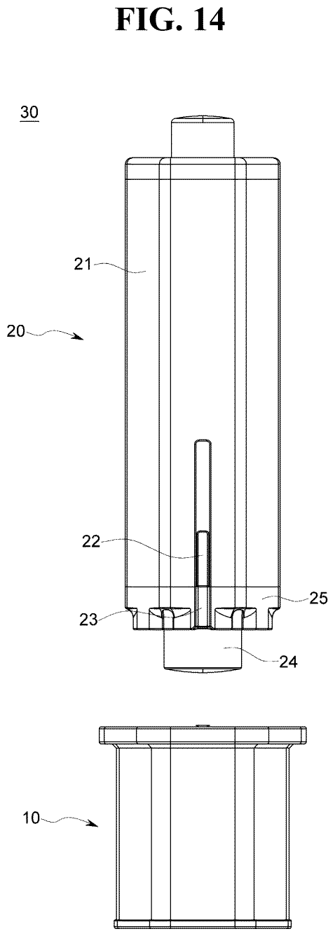

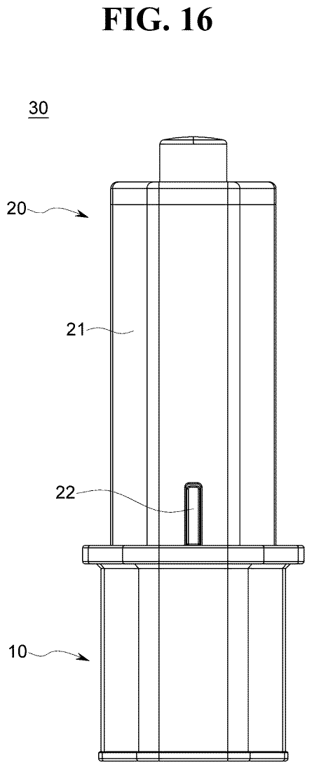

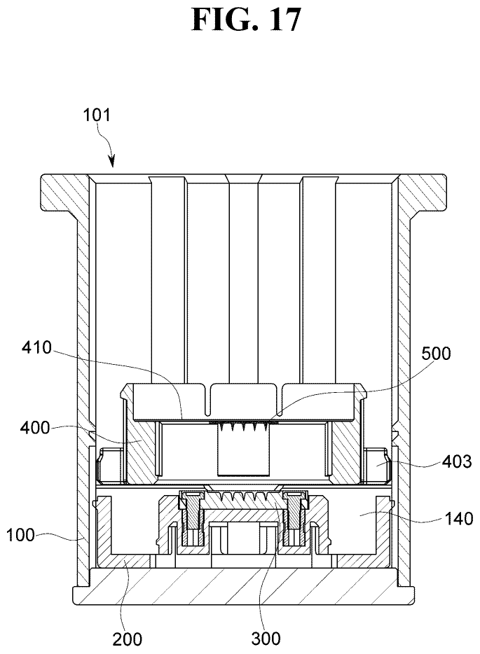

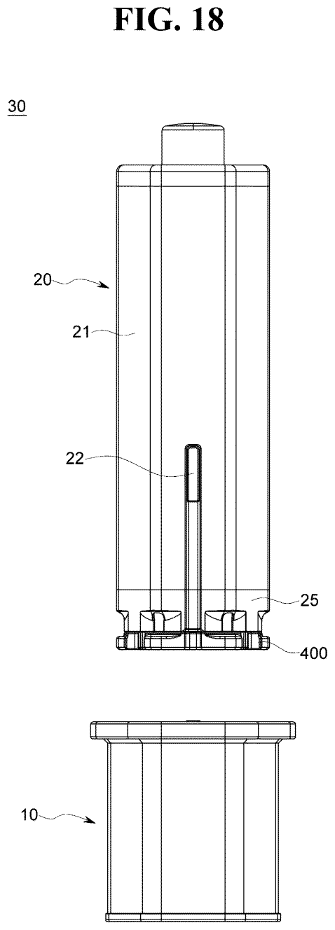

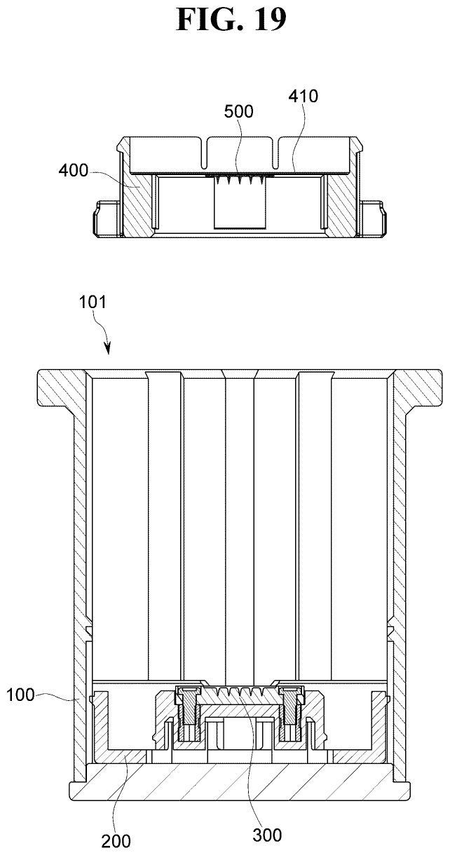

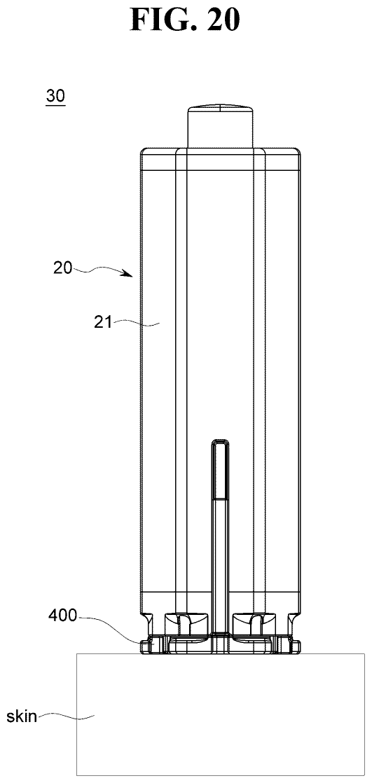

FIGS. 14, 15, 16, 17, 18, and 19 show embodiments in which a skin contact part according to an embodiment of the present disclosure is mounted on an applicator and separated from a microneedle package, wherein FIGS. 14 and 15 show a state before the applicator is mounted on the microneedle package, FIGS. 16 and 17 show a state in which the applicator is mounted on the microneedle package, and FIGS. 18 and 19 show a state in which the skin contact part is mounted on the applicator and separated from the microneedle package. At this time, FIGS. 15, 17, and 19 are for explaining the mounting positions of the mold holder and the skin contact part, and the illustration of the applicator is omitted.

The present disclosure does not require a separate action to load the firing means of the applicator 20 into a state in which firing is possible, and the skin contact part 400 is vertically detached from the microneedle package 10. This will be described in detail as follows.

Referring to FIGS. 14 and 15, the applicator 20 and the microneedle package 10 may be provided separately.

Referring to FIG. 14, the applicator 20 may approach the microneedle package 10 in a state in which the slide 22 is positioned a predetermined distance separated from the end of the applicator 21 and the pressing part 24 protrudes from the end of the applicator body 21.

Referring to FIG. 15, the microneedle cartridge 11 may be fastened to the case 100.

The microneedle cartridge 11 may be provided in a state in which it is inserted into the case opening 101 of the case 100 and fixed at a predetermined position within the case 100.

A protrusion 221 protruding outward may be provided on the case fastening part 220 of the mold holder 200. In response, a catching projection 110 may be provided on the inner side of the case 100 so that the protrusion 221 engages with the catching projection 110 to fix and couple the microneedle cartridge 11 at a predetermined position, thereby restricting rotation and length directional movement. At this time, the mold holder 200 may be detachably disposed on the catching projection 110. Accordingly, the microneedle package 10 is fixed in its regular position so that the distal end of the applicator 20 approaches the adhesive means and the base of the microneedle in a vertical direction, so that the needles in the microneedle 500 may be formed the base in a vertical direction.

The skin contact part 400 may be seated on a stepped projection 140 provided within the case 100 and thus fixed in position. That is, unlike the mold holder 200 which is detachably disposed on the catching projection 110, the skin contact part 400 may be seated on the stepped projection 140 and thus fixed in position so as not to be moved forward by the inserting applicator 20.

At this time, the skin contact part 400 and the mold holder 200 within the case 100 may be overlappingly installed within the case 100.

In addition, the microneedle package 10 of the present disclosure may be stored in a state in which the case opening 101 is closed by a sealing member (not shown) in the FIG. 15 state. Accordingly, contamination of the microneedle cartridge 11 during storage period can be prevented and storage convenience can be enhanced.

Referring to FIGS. 16 and 17, as one operation of inserting the applicator 20 into the microneedle package 10, the firing means of the applicator 20 may be loaded and simultaneously the skin contact part 400 and microneedle 500 may be fastened to the front of the applicator 20.

Referring to FIG. 16, as the applicator 20 is inserted into the case 100 of the microneedle package 10, the slide 22 and the pressing part 24 linked with the slide 22 are pulled rearward.

At this time, the slide 22 and the pressing part 24 are fixed at a position where the applicator 20 is no longer inserted into the case 100 so that the firing means is loaded, and the skin contact part 400 is fastened to the front of the applicator 20. This will be described in detail with reference to FIG. 17.

Referring to FIG. 17, when the applicator 20 is inserted, the skin contact part 400 in the case 100 may remain in its original position, but the mold holder 200 may move forward, allowing the skin contact part 400 and microneedle 500 to be separated from the mold holder 200.

For example, when the applicator 20 is inserted forward along the length direction of the case 100 into the case opening 101, the mold holder 200 is pressed by the applicator 20, causing the protrusion 221 to detach from the catching projection 110 and move forward, so that the mold holder 200 is seated on the bottom surface of the case 100. On the other hand, the skin contact part 400 is not pushed away since the tongue parts 403 are caught on the stepped projection 140.

Meanwhile, as the applicator 20 is inserted into the case 100 of the microneedle package 10, a structure in which the slide 22 is caught on the protrusion piece provided in the case 100, but the skin contact part fastening part 25 and the end of the applicator body 21 are inserted into the case 100 is applied so that the pressing part 24 is pulled rearward together with the slide 22. As another example, a structure in which the slide 22 is caught on the edge of the opening of the case 100, but the skin contact part fastening part 25 and the end of the applicator body 21 are inserted into the case 100 is applied so that the pressing part 24 may also be pulled rearward together with the slide 22.

At this time, the pressing part 24 may move to the rear together with the slide 22 so as not to touch the adhesive means 410 disposed in the skin contact part 400. Accordingly, a structure in which the needle attachment part (411 in FIG. 10) is not separated from the adhesive means 410 in advance by the pressing part 24 may be applied.

In another embodiment, a structure may be applied in which the pressing part 24 applies a predetermined pressure to the needle attachment part (411 of FIG. 10) from the adhesive means 410 disposed on the skin contact part 400 so that the microneedle 500 is sufficiently adhered to the front surface of the needle attachment part (411 of FIG. 10).

That is, the present disclosure can simultaneously perform, as one operation in which the applicator 20 is mounted on the microneedle package 10, an operation in which the slide 22 and the pressing part 24 are fixed and loaded at a predetermined position, an operation in which the microneedle 500 is detached from the mold holder 200, and an operation in which the skin contact part 400 is fastened to the front of the applicator 20.

Referring to FIGS. 18 and 19, the applicator 20 may be withdrawn from the microneedle package 10, thereby separating the applicator 20 from the microneedle package 10 in a state in which the skin contact part 400 is fastened to the skin contact part fastening part 25.

At this time, the slide 22 and the pressing part 24 have their positions fixed by the firing means. Additionally, the microneedle 500 may be attached to the front surface of the needle attachment part (411 in FIG. 10) disposed on the skin contact part 400.

In addition, as shown in FIG. 19, when the applicator 20 is withdrawn in a vertical direction from the case 100, the mold holder 200 maintains a state in which the mold holder 200 is seated on the bottom surface of the case 100 as it is, and the skin contact part 400 may be extracted to the outside of the case 100 in a state in which the skin contact part 400 is mounted on the front of the applicator 20.

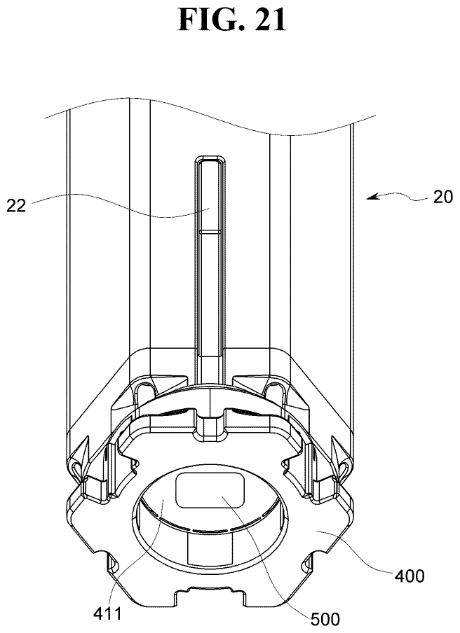

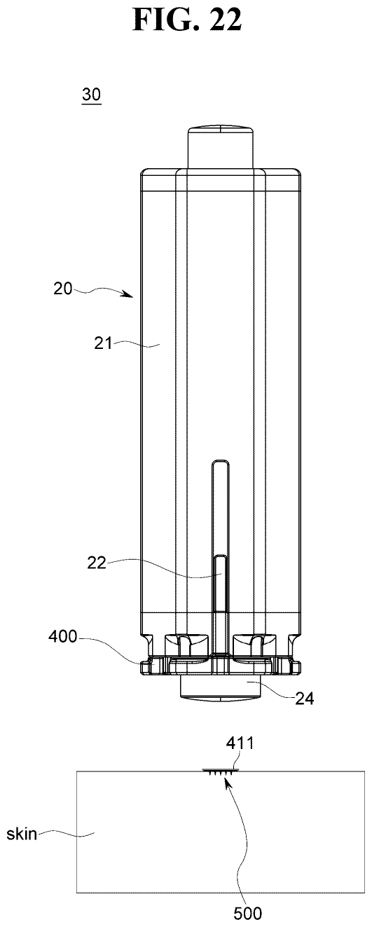

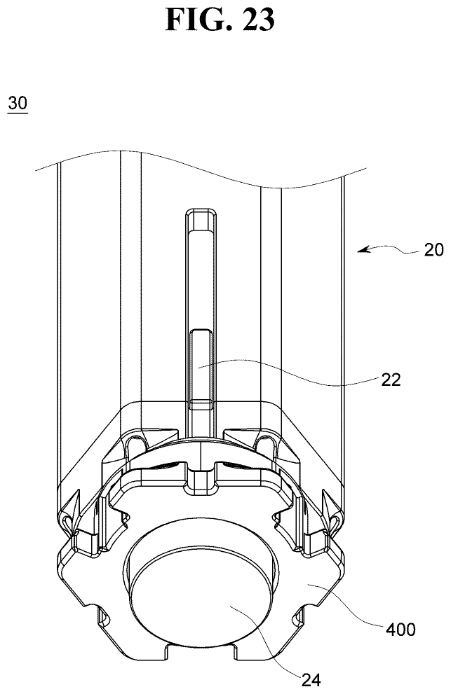

FIGS. 20, 21, 22, and 23 show embodiments in which an applicator according to an embodiment of the present disclosure contacts the skin, a microneedle is fired, and then is separated from the skin, wherein FIG. 20 shows a state in which the applicator comes into contact with the skin and is ready to fire the microneedle, FIG. 21 shows the front of the applicator in FIG. 20, FIG. 22 shows a state in which the microneedle is fired from the applicator and then is separated from the skin, and FIG. 23 shows the front of the applicator in FIG. 22, respectively.

Referring to FIGS. 20 and 21, the front surface of the skin contact part 400, for example, the front surface of the aforementioned tongue part (403 of FIG. 10), may contact the skin.

Referring to FIG. 21, since the needle attachment part 411 may be positioned further back than the front surface of the skin contact part 400, the applicator package 30 may have a structure in which the microneedle 500 does not contact the skin before the shooting button (26 of FIG. 1) is pressed after the applicator package 30 contacts the skin. Accordingly, the applicator package 30 of the present disclosure may attach the microneedle 500 to the skin at its regular position and attach it to the skin in a direction perpendicular to the skin, thereby attaching the microneedle 500 of a form formed as it is in the mold (300 of FIG. 2) to the skin.

Referring to FIGS. 22 and 23, after the microneedle 500 is fired into the skin as the pressing part 24 and the slide 22 are moved forward by the operation of the shooting button (26 of FIG. 1), the applicator 20 may be separated from the skin.

At this time, as the pressing part 24 moves forward along the length direction from the applicator 20, the needle attachment part 411 may be pressed forward so that the needle attachment part 411 is separated from the skin contact part attachment part (413 of FIG. 10) while cutting the adhesive means cutting line (412 of FIG. 10). This action may allow the microneedle 500 attached to the front surface of the needle attachment part 411 to be attached to the skin.

Thereafter, the skin contact part 400 may be separated from the applicator 20, and the applicator 20 returns to the initial state described with reference to FIG. 14, and may be coupled to another microneedle package 10.

As described above, specific description for the present disclosure has been made by embodiments with reference to the attached drawings, but since the above-described embodiments merely explain preferred examples of the present disclosure, the present disclosure should not be construed as being limited to the above-described embodiments, and the scope of rights of the present disclosure should be understood as the claims described later and concepts equivalent thereto.

For example, the drawings schematically show each component as the main body to aid understanding, and the thickness, length, number, etc. of each component illustrated may differ from the actual configuration due to the progress of drawing up of the drawing. Furthermore, the material, shape, dimensions, etc. of each component shown in the above-described embodiments are merely examples and are not particularly limited, and various modifications are possible without substantially departing from the effects of the present disclosure.

### DESCRIPTION OF REFERENCE NUMERALS

10: Microneedle package 11: Microneedle cartridge 12: Needle protection part
20: Applicator 21: Applicator body 22: Slide 23: Linear guide
24: Pressing part 25: Skin contact part fastening part 26: Shooting button 30: Applicator package
100: Case 101: Case opening 110: Catching projection 120: Guide groove 130: Protrusion piece
140: Stepped projection 200: Mold holder 210: Mold mounting part 211: Elastic piece 220: Case fastening part
221: Protrusion 300: Mold 310: Microneedle forming part 311: Base forming part
312: Needle forming part 400: Skin contact part 401a, 401b: Skin contact part penetration part
402: Case fastening part guide part 403: Tongue part 404: Tongue part groove 410: Adhesive means
411: Needle attachment part 412: Adhesive means cutting line 413: Skin contact part attachment part
420: Guide member 430: Step 500, 500a: Microneedle 510, 510a: Base
520, 520a: Needle 521: Needle centerline

1. An applicator for providing a microneedle, which comprises a slide and a pressing part that moves in conjunction with the slide, and inserts the microneedle into the skin in a vertical direction with a constant force and speed.

2. The applicator of claim 1, wherein the slide moves in a vertical direction with a constant force and speed along a linear guide formed along a length direction of the side portion of the applicator.

3. The applicator of claim 2, wherein the slide moves a preset distance from the linear guide and is fixed at both ends of the preset distance.

4. The applicator of claim 1, wherein the pressing part is at least partially inserted into the applicator and moves along a length direction of the applicator.

5. The applicator of claim 1, wherein the microneedle is disposed in front of the pressing part.

6. The applicator of claim 1, wherein the applicator has a structure that prevents rolling.

7. The applicator of claim 6, wherein the applicator includes at least one of a polygonal structure having at least one corner, an oval structure, and a rolling-prevention member.

8. An applicator package comprising:
the applicator according to claim 1; and
a microneedle package coupled to the applicator to provide the microneedle to the applicator.

9. The applicator package of claim 8, wherein the applicator is operated by including: that when the applicator is inserted into the microneedle package, the slide moves to the rear of the applicator, the pressing part moves to the rear of the applicator in conjunction therewith, and the slide and the pressing part moving backward are fixed at a predetermined position; and that the slide and the pressing part fixed at the predetermined position move forward.

10. The applicator package of claim 9, wherein the microneedle package comprises a needle protection part defining a space in which the microneedle is disposed and a case in which the needle protection part is disposed and into which the applicator is vertically inserted, the needle protection part comprises a mold holder that is pushed by the applicator and moves forward and a skin contact part that is fastened to the front of the applicator, and the slide and the pressing part are fixed at a predetermined position and the skin contact part is fastened to the front of the applicator.

11. The applicator package of claim 10, wherein as a single operation of mounting the applicator on the microneedle package, an operation of fixing and loading the slide and the pressing part at a predetermined position, an operation of removing the mold holder, and an operation of fastening the skin contact part to the front of the applicator are performed.

12. The applicator package of claim 9, wherein the case of the microneedle package is provided with a guide means at the inner side, so that as the applicator is inserted into the case, the slide moves rearward by the guide means, and accordingly, the pressing part moves rearward, so that the pressing part does not have to be pushed in directly, thereby preventing contamination of the pressing part and the microneedle.

13. The applicator package of claim 8, wherein the microneedle package comprises a needle protection part defining a space in which the microneedle is disposed, and a case in which the needle protection part is disposed and into which the applicator is vertically inserted.

14. The applicator package of claim 13, wherein the needle protection part comprises a mold holder that is pushed by the applicator and moves forward and a skin contact part that is fastened to the front of the applicator.

15. The applicator package of claim 14, wherein the mold holder includes a case fastening part that is secured to a predetermined position within the case.

16. The applicator package of claim 15, wherein the case fastening part is pressed by the applicator, thereby moving the mold holder forward.

17. The applicator package of claim 16, wherein the case fastening part is provided with three pressed regions that are pressed by the applicator, and the mold holder is pressed by the applicator such that the mold holder has a surface perpendicular to the applicator, thereby forming the microneedle vertically.

18. The applicator package of claim 8, wherein the slide is moved backward by a guide member provided in the microneedle package.

19. The applicator package of claim 18, wherein the applicator further comprises a linear guide provided to guide the slide to move in a vertical direction with a constant force and speed, and the guide member is inserted into the linear guide as the applicator is inserted into the microneedle package.
